(19) United States
(12) Patent Application Publication
(10) Pub. No.: US 2011/0015743 A1
(43) Pub. Date: Jan. 20, 2011

(54) MULTI-DENSITY POLYMERIC INTERBODY SPACER

(75) Inventors: Richard J. Deslauriers, Woodbury, CT (US); Joseph Jannetty, Naugatuck, CT (US); Eric Kolb, Sandy Hook, CT (US); John A. Tomich, Wallingford, CT (US); Naresh Akkarapaka, West Haven, CT (US)

Correspondence Address: MCCORMICK, PAULDING & HUBER LLP, CITY PLACE II, 185 ASYLUM STREET, HARTFORD, CT, 06103, US

(73) Assignee: DOCTORS RESEARCH GROUP, INC., Southbury, CT (US)

(21) Appl. No.: 12/502,597

(22) Filed: Jul. 14, 2009

(51) Int. Cl. A61F 2/44

(52) U.S. Cl. 623/17.16

(57) ABSTRACT

A multi-density polymeric interbody spacer formed from biocompatible material for osteoconductivity includes multiple density regions of different porosity to provide both strength and osteoconductivity. An interface region is formed between the density regions to provide both direct adhesion and mechanical interlocking between the different density regions to increase the strength of the multi-density polymeric interbody spacer. A method for forming the multi-density polymeric interbody spacer includes curing a first density region to achieve a first target porosity. A second density region may then be molded to the first density region to achieve a second target porosity. A portion of the second density region partially flows into pores of the first density region, providing direct adhesion and mechanical interlocking between the first and second density regions.

### FIELD OF THE INVENTION

The present invention relates to implants for use in interbody fusion and methods of manufacturing such implants and, more particularly, to implants formed from synthetic bone polymers.

### BACKGROUND OF THE INVENTION

There are many situations in which bones or bone fragments are fused, including fractures, joint degeneration, abnormal bone growth, infection and the like. For example, circumstances requiring spinal fusion include degenerative disc disease, spinal disc herniation, discogenic pain, spinal tumors, vertebral fractures, scoliosis, kyphosis, spondylolisthesis, spondylosis, Posterior Rami Syndrome, other degenerative spinal diseases, and other conditions that result in instability of the spine.

During spinal surgical procedures, a discectomy or corpectomy may be performed to remove an intervertebral disc or a vertebral body or portion thereof. It is known to implant interbody spacers to replace the removed intervertebral disc or vertebral body to restore height and spinal stability.

Conventional interbody spacers have been formed through autograft procedures, removing bone from a patient's iliac crest for use as an interbody spacer. However, autograft procedures are disadvantageous since they require a second operative site with associated pain.

Another form of interbody spacer used for spinal fusion is a machined allograft interbody spacer, which is formed from bone transplanted from another person, typically a cadaver. Thus, machined allograft interbody spacers are advantageous because they eliminate the need for the second operative site. However, machined allograft interbody spacers have other drawbacks that make them undesirable for spinal fusion applications. For example, there is a limited supply of qualified bone that can be formed into machined allograft interbody spacers, which results in increased cost and product backorder. Also, the size and shape of available qualified bone limits the size of machined allograft spacers. Additionally, to be qualified, the transplanted bone must be tested for disease and undergo expensive sterilization to reduce the risk of disease transmission. However, even with testing and sterilization, the risk of disease transmission cannot be completely eliminated. The cadaver bone must also be manufactured into the proper spacer geometry for the machined allograft interbody spacer since the transplanted cadaver bone cannot exactly match the disk being removed from a patient. The varied quality of source bone also makes it challenging to maintain uniform mechanical properties of allograft interbody spacers. Some allograft multiple bone density spacers may be cut as a single piece from cadaver bone, for example, from the femur bone. However, a cadaver will likely only produce a few such spacers since there are a very limited number of bone sources to produce a sufficient geometry of sufficient cortical and cancellous bone. Thus, allograft interbody spacers are typically assembled from multiple bone density regions, which requires the additional manufacturing of a mechanical interlock, such as a pin feature or a dovetail feature, between the parts of the multipart spacer, thereby increasing cost of manufacturing.

Interbody spacers have also been formed from non-bone material as hollow rigid structures, for example, from metal or polyaryletheretherketone (PEEK). These hollow rigid spacers have many deficiencies. For example, metal spacers are too stiff to share the load across the vertebrae and PEEK is very brittle. Rigid spacers formed from metal or PEEK also fail to provide a structure for osteoconduction. Thus, if osteoconduction is desired, a secondary material is required to act as an osteoconductive scaffold. Additionally, hollow rigid spacers may result in vertebrae getting crushed due to their stiffness. Hollow rigid spacers formed from metal also require a relatively significant amount of machining, increasing manufacturing complexity.

Interbody spacers have also been formed from composite synthetic structures using heat to expand and contract metal tube over porous ceramic structure. These have the same disadvantage of hollow rigid structures formed of metal in that they are too stiff to share the load.

Single density interbody spacers formed from polyurethanes have also been manufactured for spinal fusion applications. Polyurethanes are advantageous for orthopedic applications because fillers, such as calcium phosphate or calcium carbonate, can be incorporated into the polyurethane to form a more porous structure through resorption, which allows a targeted porosity for osteoconduction to be achieved. However, while the porous polyurethane structure is ideal for osteoconduction, polyurethane interbody spacers formed with a porous structure lack the strength to withstand the forces seen after spinal fusion.

Accordingly, there a need for an interbody spacer that promotes bone growth with appropriate strength and structure for interbody fusion applications.

### SUMMARY OF THE INVENTION

According to the present invention, a multi-density polymeric interbody spacer is a synthetic spacer that may be implanted to restore height and promote bone fusion after discectomy or corpectomy. The multi-density polymeric interbody spacer is formed from biocompatible polymeric foam for osteoconductivity, preferably a polyurethane-urea. The multi-density structure provides for combined strength and porosity. The multi-density spacer includes direct adhesion and mechanical interlocking between different density regions to increase the strength of the interbody spacer. The multi-density spacer may also include geometric surface features to enhance positioning and fit of the spacer.

According to one embodiment of the present invention, the multi-density polymeric interbody spacer has a second density region of high density surrounding a less dense core first density region and a spacer perimeter surface with a predetermined shape suitable for a desired application.

According to another embodiment of the present invention, the multi-density polymeric interbody spacer includes a central first density region of lower density and two lateral second density regions of greater density adjacent to the central first density region.

According to the present invention, a method for forming a multi-density polymeric interbody spacer includes curing the first density region of lower density in a vacuum to achieve a target porosity. The cured first density region may be machined to achieve a desired shape, for example a cylinder or a rectangular shape. The second density region or regions of greater density may then be molded, under pressure, to the first density region of lower density. A portion of the region of greater density partially flows into the pores of the first density region of lower density, to form an interface region providing direct adhesion and porous interlocking between the first density region of lower density and the second density region or regions of greater density. The multi-density polymeric interbody spacer may then be machined to achieve a desired final shape or to add geometric features to enhance positioning and fit of the spacer.

According to the present invention, multiple multi-density polymeric interbody spacers may be molded as a single multi-density polymeric volume. The multi-density polymeric interbody spacers are then cut from the multi-density polymeric volume.

According to the present invention, the second density region may be formed in a closed mold to achieve the second pressure.

According to the present invention, the multi-density polymeric interbody spacer is molded between first and second platens. The orientation of the first and second platens is changed during the curing process to impart the multi-density polymeric interbody spacer with anisotropic material properties.

These and other objects, features and advantages of the present invention will become apparent in light of the following detailed description of non-limiting embodiments, with reference to the accompanying drawings.

### BRIEF DESCRIPTION OF THE DRAWINGS

FIG. 1 is a perspective view of a multi-density polymeric interbody spacer according to an embodiment of the present invention;

FIG. 2 is a perspective view of another embodiment of the multi-density polymeric interbody spacer;

FIG. 3 is a cross-sectional view of the multi-density polymeric interbody spacer of FIG. 2;

FIG. 4 is a cross-sectional view of the multi-density polymeric interbody spacer according to FIG. 1 implanted between vertebrae;

FIG. 5 is a perspective view of another embodiment of the multi-density polymeric interbody spacer of FIG. 2;

FIG. 6 is an enlarged cross-sectional view of a portion of an interface region of the multi-density polymeric interbody spacer of FIG. 4;

FIG. 7 is an enlarged cross-sectional view of another embodiment of the interface region of FIG. 4;

FIG. 8 is a cross-sectional view of a multi-density polymeric interbody spacer according to another embodiment of the present invention;

FIG. 9 is a process diagram showing a method of making the multi-density polymeric interbody spacer of FIG. 1;

FIG. 10 is a perspective view of a second density region of another embodiment of the multi-density polymeric interbody spacer;

FIG. 11 is a perspective view of a first density region of another embodiment of the multi-density polymeric interbody spacer;

FIG. 12 is a perspective view of another embodiment of the multi-density polymeric interbody spacer;

FIG. 13 is a process step for fabricating a plurality of the multi-density polymeric interbody spacers of FIG. 1;

FIG. 14 is a perspective view of another embodiment of the multi-density polymeric interbody spacer;

FIG. 15 is a cross-sectional view of the multi-density polymeric interbody spacer of FIG. 14;

FIG. 16 is a perspective view of another embodiment of the multi-density polymeric interbody spacer;

FIG. 17 is a process step for fabricating a plurality of the multi-density polymeric interbody spacers of FIG. 16;

FIG. 18 is a perspective view of another embodiment of the multi-density polymeric interbody spacer;

FIG. 19 is a perspective view of another embodiment of the multi-density polymeric interbody spacer;

FIG. 20 is a process diagram showing a method of implanting the multi-density polymeric interbody spacer of FIG. 19;

FIG. 21 is a perspective view of another embodiment of the multi-density polymeric interbody spacer;

FIG. 22 is a process diagram showing another method of making the multi-density polymeric interbody spacer of FIG. 1;

FIG. 23 is a process diagram showing another method of forming a first density region of the multi-density polymeric interbody spacer of FIG. 1;

FIG. 24 is a cross-sectional view of a biocompatible polymeric material of FIG. 23;

FIG. 25 is a process diagram showing another method of forming the multi-density polymeric interbody spacer of FIG. 1;

FIG. 26 is a process diagram showing another method of forming the multi-density polymeric interbody spacer of FIG. 1;

FIG. 27 is a cross-sectional view of another embodiment of the multi-density polymeric interbody spacer;

FIG. 28 is a process step of another embodiment for fabricating the the multi-density polymeric interbody spacer;

FIG. 29 is a cut-away perspective view of another embodiment of the multi-density polymeric interbody spacer; and

FIG. 30 is a perspective view of another embodiment of the multi-density polymeric interbody spacer.

### DETAILED DESCRIPTION OF A PREFERRED EMBODIMENT

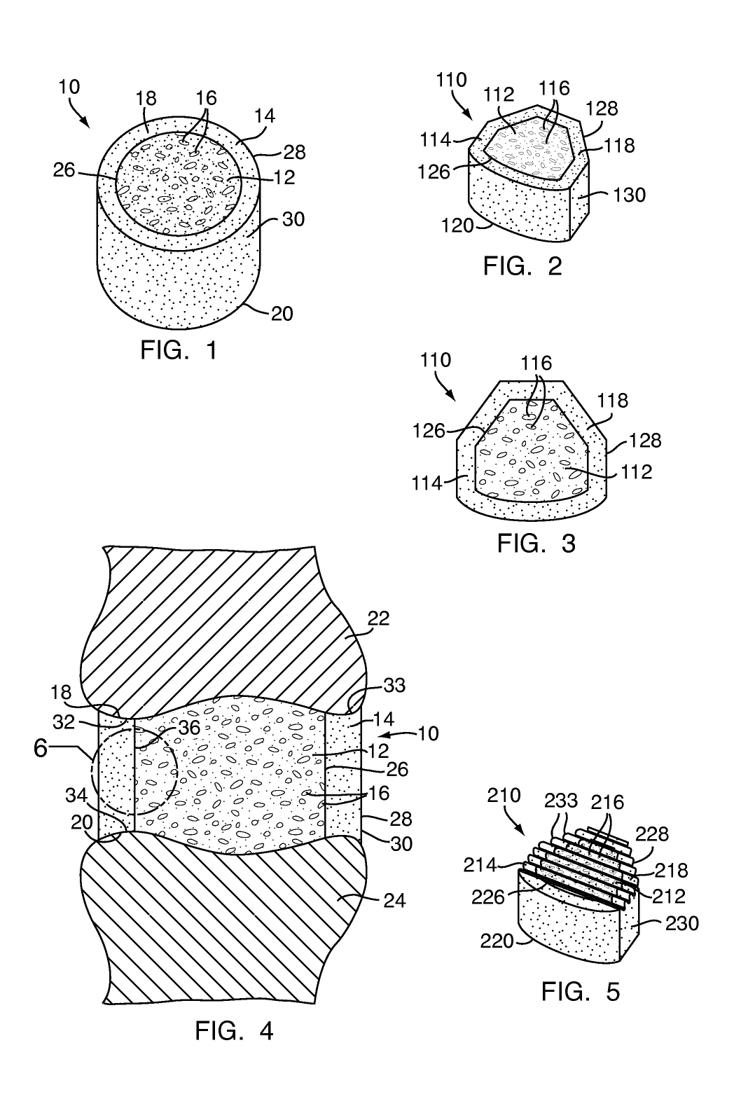

Referring to FIG. 1, a multi-density polymeric interbody spacer 10, for replacing an intervertebral disc in spinal fusion surgery to restore height and promote bone fusion, includes a first density region 12 forming a central core and a second density region 14 surrounding the first density region 12. The first density region 12 and the second density region 14 are formed from a biocompatible polymeric foam material, which is discussed below in greater detail. The first density region 12 is formed to have a low density and a high porosity, providing a porous structure with pores 16 to allow bony ingrowth or osteoconduction after implantation of the multi-density polymeric interbody spacer 10 during spinal fusion surgery. The second density region 14 has a relative high density with low porosity to provide the multi-density polymeric interbody spacer 10 with strength to withstand spinal fusion forces, which, for example, may be in the vicinity of two thousand Newtons (2000N) for cervical vertebrae spacers or even larger for thoracic and lumbar spacers. Additionally, the strength of the second density region 14 may be as much as one hundred times the strength of the first density region 12. The multi-density polymeric interbody spacer 10 has a superior surface 18 and an inferior surface 20 for contacting, after implantation, a first vertebra 22 and a second vertebra 24, respectively, as shown in FIG. 4.

The first density region 12 has a defined first region perimeter surface 26, which extends from the superior surface 18 to the inferior surface 20. The second density region 14 also extends from the superior surface 18 to the inferior surface 20 and substantially surrounds the first region perimeter surface 26 of the first density region 12. The second density region 14 has a defined second region perimeter surface 28, which in this embodiment corresponds to a spacer perimeter surface 30 of the multi-density polymeric interbody spacer 10.

Although shown as having substantially cylindrical first region, second region and spacer perimeter surfaces 26, 28, 30, each perimeter surfaces will have a predetermined shape suitable for a desired spacer application. For example, referring to FIGS. 2 and 3, wherein like numerals represent like features, the first region perimeter surface 126, second region perimeter surface 128 and spacer perimeter surface 130 may be substantially trapezoidal in shape. Alternatively, the first region perimeter surface 126 of the first density region 112 may be substantially cylindrical and be surrounded by the second density region 114 having a substantially trapezoidal second region perimeter surface 128. Furthermore, first region, second region and spacer perimeter surfaces 126,128, 130 can be of any shape needed for a particular application.

Referring to FIG. 4, the multi-density polymeric interbody spacer 10 is shown implanted between the first vertebra 22 and the second vertebra 24. The superior surface 18 of the multi-density polymeric interbody spacer 10 is designed to substantially match the geometric shape of a first vertebral end plate 32 of the first vertebra 22 and may include surface features 33, for example, by machining angled wedges and/or ramps into the superior surface 18. Similarly, surface features may be added to the inferior surface 20 of the multi-density polymeric interbody spacer 10 to substantially conform the inferior surface 20 to the shape of a second vertebral end plate 34 of the second vertebra 24.

Referring to FIG. 5, the superior and inferior surfaces 218 and 220 of the multi-density polymeric interbody spacer 210 may include surface features 233 to improve fit between and contact with the first and second vertebrae 22, 24, shown in FIG. 4. The surface features 233 may include wedges, ramps, spikes, ridges or any combination thereof. Additionally, the surface features 233 will minimize spacer migration after implantation.

Referring back to FIG. 1, as discussed above, the first density region 12 and the second density region 14 of the multi-density polymeric interbody spacer 10 are formed from a biocompatible polymeric rigid foam material, which promotes bone growth when used in medical procedures. Preferably, the biocompatible polymeric foam material is foam formed from a polyurethane/polyurea such as the KRYPTONITE™ bone matrix product, available from DOCTORS RESEARCH GROUP, INC. of Southbury, Conn., and also described in U.S. patent application Ser. No. 11/089,489, which is hereby incorporated by reference in its entirety. The biocompatible polymeric material is initially prepared in a liquid state. When cured, the biocompatible polymeric material will pass through a taffy-like state, in which the biocompatible polymeric material is easily malleable and may be shaped and sculpted to a desired geometry. The biocompatible polymeric material then cures into a final solid state.

The biocompatible polymeric material may combine an isocyanate with one or more polyols and/or polyamines, along with optional additives (e.g., water, filler materials, catalysts, surfactants, proteins, and the like), permitting the materials to react to form a composition that comprises biocompatible polyurethane/polyurea components. As referred to herein, the term “biocompatible polyurethane/polyurea components” includes, inter alia, biocompatible polyester urethanes, biocompatible polyether urethanes, biocompatible poly(urethane-ureas), biocompatible polyureas, and the like, and mixtures thereof.

Certain embodiments may comprise biocompatible polyurethane/polyurea components present in an amount in the range of from about twenty percent to about ninety percent (20% to about 90%) by weight of the composition, with the balance comprising additives. Certain embodiments of the compositions made according to the present invention may comprise biocompatible polyurethane/polyurea components present in an amount in the range of from about fifty percent to about eighty percent (50% to about 80%) by weight of the composition, with the balance comprising additives.

The biocompatible compositions may also combine an isocyanate prepolymer with a polyol or chain-extender, and a catalyst, along with optional additives (e.g., filler material), permitting them to react to form a composition that comprises biocompatible poly(urethane-isocyanurate) components. In certain embodiments, the isocyanate prepolymer may react with a polyol, water, and a catalyst to form a composition that comprises biocompatible poly(urethane-urea-isocyanurate) components; optional additives also may be included in the composition.

Preferably, the first density region 12 and the second density region 14 have the same material composition, with the only difference being the region's density and, conversely, porosity. Producing the first density region 12 and the second density region 14 from a single material composition provides for strong direct adhesion between the first and second density regions 12, 14. Additionally, the single material composition eliminates the need for proving biocompatibility of multiple materials. However, the multi-density polymeric interbody spacer 10 according to the present invention may be formed with first and second density regions 12, 14 having different material compositions that are each biocompatible, if desired. For example, the first density region 12 may include an additional surfactant to increase interconnectivity of pores 16 and the second density region 14 may include less water to minimize formation of carbon dioxide bubbles during polymerization.

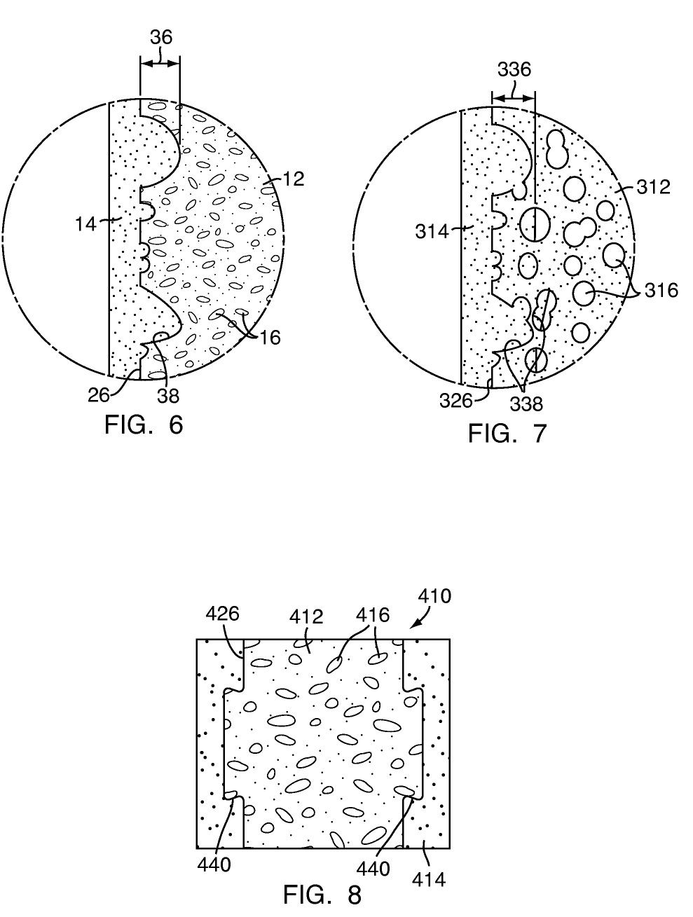

Referring to FIG. 6, the multi-density polymeric interbody spacer 10 includes an interface region 36 connecting the first density region 12 to the second density region 14. The first density region 12 and the second density region 14 may be connected to one another through direct adhesion in the interface region 36; for example, by adhesive properties of the first density region 12, the second density region 14 or both. Direct adhesion as used herein includes adsorption, chemical bonding and/or diffusion or any other method of adhesion know to one skilled in the art. Additionally, the interface region 36 may include mechanical interlocking in the form of a porous interlocking 38, which forms a mechanical connection between the first density region 12 and the second density region 14. The porous interlocking 38 is formed by a portion of the second density region 14 that occupies pores 16 located around the first region perimeter surface 26 of the first density region 12. Preferably, the interface region 36 is less than one millimeter (1 mm) in thickness.

Although shown in FIG. 6 as having a primarily closed pore cell structure, the first density region 312 may instead, more preferably, have an open pore cell structure as shown in FIG. 7 by providing more interconnectivity between pores 316. The open pore cell structure with increased pore interconnectivity provides for strong mechanical interlocking through porous interlocking 338.

Referring to FIG. 8, the multi-density polymeric interbody spacer 410 may also include macro features 440, for example, a dovetail feature or a pin feature, to provide an additional or alternative mechanical interlock between the first density region 412 and the second density region 414. Thus, the multi-density polymeric interbody spacer 410 may include the porous interlocking, the mechanical interlock or a combination of both the porous interlocking and the mechanical interlock. The macro features 440 may be formed to extend from a perimeter surface 426 of the first density region 412, as shown. Alternatively, the macro features 440 may be formed as cavities in the perimeter surface 426 of the first density region 412, into which a portion of the second density region 414 is able to penetrate.

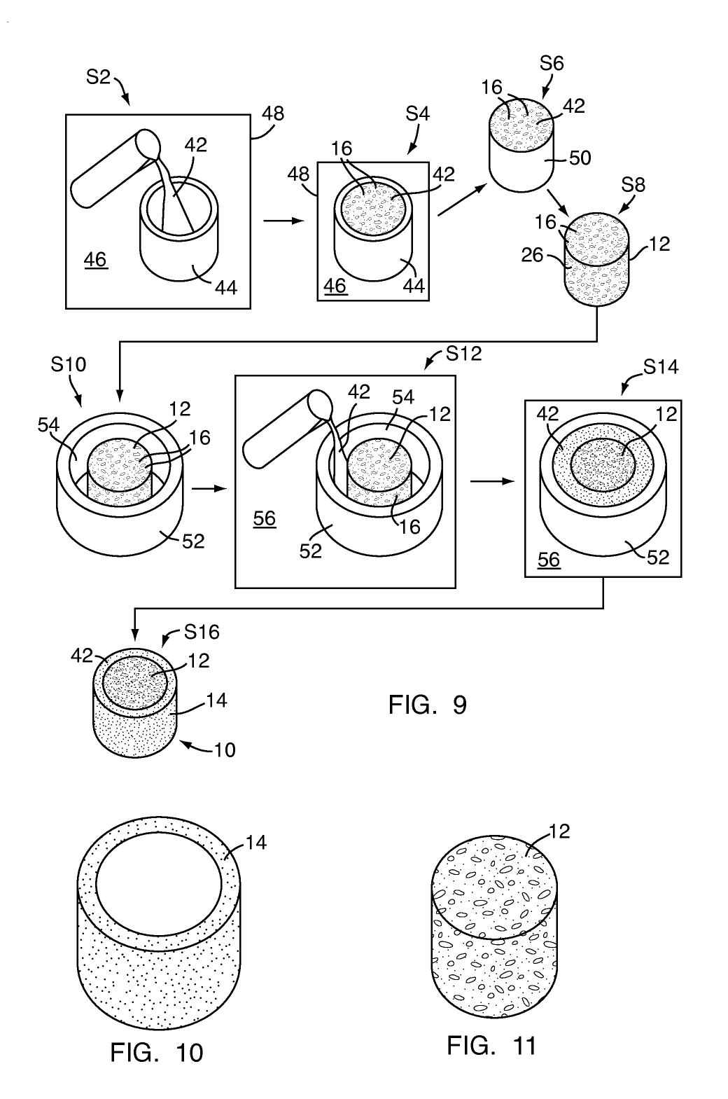

Referring to FIG. 9, a method of forming the multi-density polymeric interbody spacer 10 using varying pressures to affect porosity is shown. In step S2, a biocompatible polymeric material 42, in a liquid state, is poured into a first mold 44 at a first pressure 46. As discussed above, the biocompatible polymeric material 42 is preferably the KRYPTONITE™ bone matrix product, available from DOCTORS RESEARCH GROUP, INC. of Southbury, Conn. The first pressure 46 may be established, for example, by placing the first mold 44 in a vacuum chamber 48 to create a low-pressure environment. Although shown in step S2 as being subjected to the first pressure 46 prior to pouring, the biocompatible polymeric material 42 may instead be poured into the first mold 44 and then subjected to the first pressure 46, for example, by placing the filled first mold 44 in a vacuum chamber 48.

In step S4, the biocompatible polymeric material 42 is maintained at the first pressure 46 and allowed to polymerize, which results in off-gassing of carbon dioxide byproducts, to form the first density region 12. In the low-pressure environment, the carbon dioxide byproducts of the polymerization process expand and form large pores 16 with a high degree of pore interconnectivity in the first density region 12. Preferably, the first pressure 46 is in the range of approximately ten inches of mercury to thirty inches of mercury (10″ Hg to 30″ Hg) to produce the first density region 12 having approximately sixty percent to ninety percent (60%-90%) porosity. All pressures are gauge pressures relative to atmospheric pressure. As discussed above, the first pressure 46 is preferably selected to provide high pore interconnectivity by allowing for a high degree of carbon dioxide cell rupture during polymerization, resulting in pores 16 that are interconnected. The low first pressure 46 makes it possible to form an open cell structure within a biocompatible polymeric material 42 that would have a substantially closed pore structure at ambient pressure. Although the first pressure 46 is preferably a vacuum, the first pressure 46 may be any other pressure capable of forming the desired porosity of the first density region 12, including ambient pressure. Once the biocompatible polymeric material 42 has fully cured, the pores 16 remain within the first density region 12 upon removal from the low-pressure environment.

In step S6, the fully polymerized biocompatible polymeric material 42 is removed from the first mold 44. When removed from the first mold 44, the fully polymerized biocompatible polymeric material 42 may include a skim coat 50 around its perimeter surface, which may result from the molding process. The skim coat 50 is a smooth layer of biocompatible polymeric material 42, formed on the perimeter surface, with substantially no pores and is typically less than one millimeter (1 mm) thick. In step S8, the skim coat 50, if present, is removed from the molded biocompatible polymeric material 42, for example by cutting or blasting, from the first density region 12 and to expose pores 16 around the first region perimeter surface 26 of the first density region 12. The molding process results in near net production of the first density region 12, thereby obviating or minimizing post molding machining. However, if necessary, the first density region 12 may be machined to the proper and/or desired final shape, for example, from a larger block of the molded biocompatible polymeric material 42.

Other known methods of increasing porosity in a primarily closed cell porous structure to form a relatively open cell porous structure may also be implemented to produce the first density region 12. For example, as an alternative to curing the biocompatible polymeric material 42 in the low-pressure environment to form pores 16 with a high degree of interconnectivity, the desired porosity of the first density region 12 may instead be formed by reticulation, which uses gases to cause internal explosions that blow out foam material, leaving an open cell porous structure behind. Alternatively, additives such as water and surfactants may be used to affect polymerization and alter porosity.

One skilled in the art would also know various methods of eliminating the skim coat 50 from forming so that the first density region 12 may be cast directly with a porous first region perimeter surface 26, eliminating the skim coat 50. For example, to cast the first density region 12 with a porous first region perimeter surface 26, the first mold 44 may be coated with a powdered or granulated biocompatible polymeric material prior to filling the first mold 44 with the liquid biocompatible polymeric material 42. Once the fully polymerized biocompatible polymeric material 42 is removed from the first mold 44 in step S6, the powdered biocompatible polymeric material may be easily removed, leaving a porous or pitted outer surface behind. Likewise, the granulated material may be partially encapsulated in the surface, thereby leaving ample voids in the skim coat to promote osseointegration. Preferably, the powdered or granulated biocompatible polymeric material has the same material composition as biocompatible polymeric material 42.

In step S10, the first density region 12 is positioned in a second mold 52 that provides space 54 for molding the second density region 14. In step S12, biocompatible polymeric material 42, in liquid state, is added to the second mold 52 at a second pressure 56 to fill space 54. The liquid state biocompatible polymeric material 42 is able to flow and expand into the pores 16 formed on the first region perimeter surface 26 of the first density region 12. Although shown in step S12 as being subjected to the second pressure 56 when added, the biocompatible polymeric material 42 may instead be added to the second mold 52 and then subjected to the second pressure 56. In step S14, the biocompatible polymeric material 42 is maintained at the second pressure 56 and allowed to polymerize to form the second density region 14. Carbon dioxide byproducts of the polymerization process again expand to form pores in the second density region 14. However, since the second pressure 56 is greater than the first pressure 46, the carbon dioxide will produce smaller pores, resulting in a second density region 14 with a lower porosity and, conversely, a higher density than the first density region 12. Additionally, since the liquid biocompatible polymeric material 42 is able to flow into the pores 16 of the first density region 12 during step S12, the biocompatible polymeric material 42 cures in the pores 16 during step S14 to form the porous interlocking 38. Preferably, the second pressure 56 is in the range of approximately five pounds per square inch to twenty pounds per square inch (5 psi-20 psi) to produce the second density region 14 having less than approximately fifty percent (50%) porosity. However, the second pressure 56 may be any pressure capable of forming the desired porosity of the second density region 14.

In step S16, the fully polymerized biocompatible polymeric material 42 and the connected first density region 12 are removed from the second mold 52 and the polymerized biocompatible polymeric material 42 is machined to the proper shape of the second density region 14, if necessary, to form the multi-density polymeric interbody spacer 10.

The present invention has been described as implementing the lower first pressure 46 to fabricate the high porosity first density region 12 in the form of a core and implementing the relatively high second pressure 56 to fabricated the low porosity second density region 14 to surround the high porosity first density region 12. However, as should be understood by those skilled in the art, the lower first pressure 46 may instead be used to fabricate a high porosity outer first density region 12 and the second pressure 56 used to form the core low porosity second density region 14.

Forming the first density region 12 with a higher porosity prior to forming the second density region 14 with a lower porosity is advantageous because larger and more numerous pores 16 are formed on the first region perimeter surface 26, providing for a strong porous interlocking 38. However, if a weaker porous interlocking 38 is acceptable, the lower porosity region may instead be formed prior to the higher porosity region according to the same process of FIG. 9. Additionally, macro features 440, discussed above in connection with FIG. 8, may be included to provide additional strength to the interface region 36.

Referring to FIG. 10, in one embodiment, it may be desirable to provide a user with only the lower porosity second density region 14, e.g. in the form of a hollow ring. The user then adds biocompatible polymeric material 42 into the ring to form the lower density first density region 12 during spinal surgery to achieve a strong bond between not only the first density region 12 and the second density region 14, but also between the multi-density polymeric interbody spacer 10 and the first and second end plates 32, 34.

Similarly, Referring to FIG. 11, the user may be provided with only the high porosity first density region 12, i.e. in the form of a cylindrical core. During surgery, the user adds the biocompatible polymeric material 42, in taffy-like form as discussed above, around the first density region 12 to form the second density region 14. This allows the user to shape a customized spacer perimeter surface 30 and customized geometry for the multi-density polymeric interbody spacer 10.

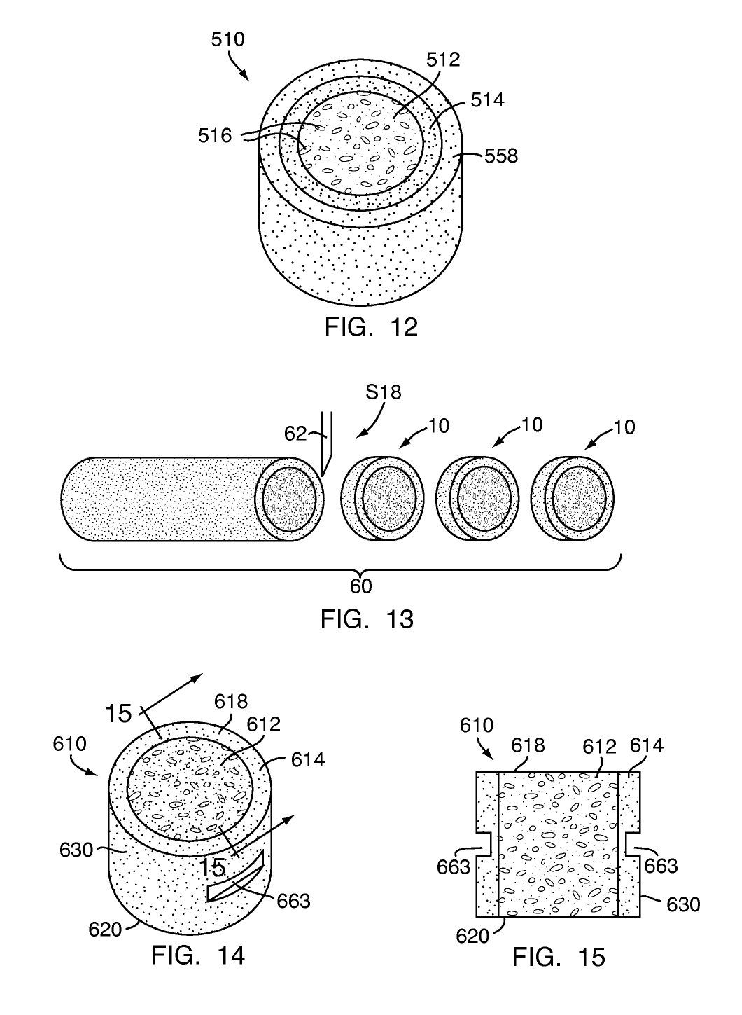

As should be understood by those skilled in the art, the process described in connection with FIG. 9 may be repeated at various pressures to create multi-density polymeric interbody spacers with additional density regions. For example, referring to FIG. 12, multi-density polymeric interbody spacer 510 may include a third density region 558 in addition to the first density region 512 and the second density region 514. The third density region 558 is formed according to same process of FIG. 9 with polymerization occurring at a third distinct pressure to achieve a different porosity from the first density region 512 and the second density region 514. The third density region 558 may also be formed with the same porosity as the first density region 512 to form the multi-density polymeric interbody spacer 510 with regions of alternating porosity. Additional density regions may be formed in the same manner to provide the multi-density polymeric interbody spacer 510 with any desired number of regions of alternating or differing porosities. Forming the multi-density polymeric interbody spacer 510 with a relatively low density external third density region 558 may speed bone ingrowth in applications where osteoclasts and osteoblasts migrate from the exterior surfaces of the host bone. The relatively porous exterior also provides a structure to which secondary osteoconductive or osteoinductive agents can be more readily added and retained.

Referring to FIGS. 9 and 13, a plurality of multi-density polymeric interbody spacers 10 may be fabricated according to the process of FIG. 9 by forming elongated first and second molds 44, 52. For example, the first and second molds 44, 52 may be ten (10) times the desired length of a single multi-density polymeric interbody spacer 10. The elongated first and second molds 44, 52 produce a multi-density polymeric volume 60. Once the multi-density polymeric volume 60 has been formed, the process of FIG. 9 includes an additional final step S18, shown in FIG. 13, to cut the multi-density polymeric interbody spacers 10 from the multi-density polymeric volume 60 using a cutting tool 62.

Although the multi-density polymeric interbody spacer 10 of FIG. 1 is shown with first region perimeter surface 26 and second region perimeter surface 28 as substantially cylindrical, the shape and geometry of the multi-density polymeric interbody spacer 10 may be varied to balance a variety of factors including implant strength, osteoconductive potential, ease of implantation, anatomic fit and user familiarity to currently available products. For example, referring to FIGS. 14 and 15, the multi-density polymeric interbody spacer 610 may include lateral slots 663 for mating with an insertion tool (not shown) to ease implantation and handling of the multi-density polymeric interbody spacer 610.

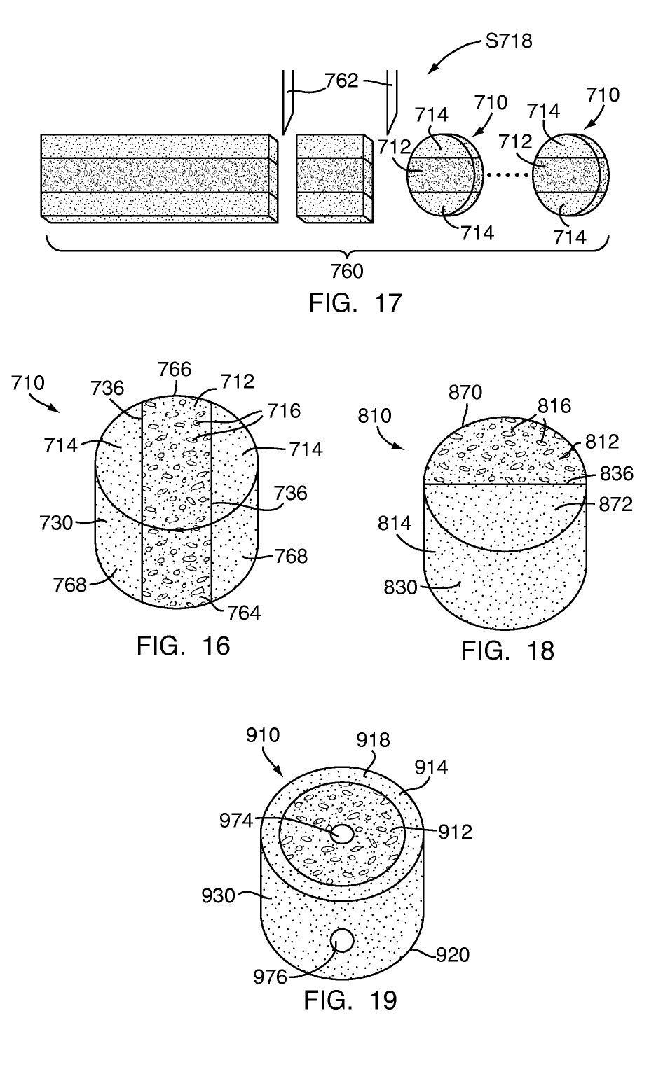

Referring to FIG. 16, in an alternative embodiment, the multi-density polymeric interbody spacer 710 includes first density region 712 having a high porosity, and two second density regions 714 having low porosity. Similar to the previously discussed embodiment, the spacer perimeter surface 730 of the multi-density polymeric interbody spacer 710 is substantially cylindrical. However, in this embodiment, the second density region 714 does not completely surround the first density region 712. Instead, the first density region 712 extends through the medial region of the multi-density polymeric interbody spacer 710 having an anterior surface 764 and a posterior surface 766 that form a portion of the spacer perimeter surface 730. The multi-density polymeric interbody spacer 710 includes two second density regions 714 disposed laterally on either side of the medial first density region 712. The second density regions 714 include lateral edges 768 that form the remainder of the spacer perimeter surface 730. The multi-density polymeric interbody spacer 710 has two interface regions 736, each formed between one of the second density regions 714 and the adjacent edge of the first density region 712.

The multi-density polymeric interbody spacers 710 may be formed according to the same process discussed in connection with FIGS. 9 and 13. Additionally, referring to FIG. 17, multi-density polymeric interbody spacers 710 having the medial first density region 712 and two laterally disposed second density regions 714 may be formed in elongated rectangular first and second molds (not shown) to produce rectangular multi-density polymeric volume 760. The multi-density polymeric volume 760 is then cut and shaped to form the multi-density polymeric interbody spacers 710 using one or more cutting tools 762 in additional step S718.

Referring to FIG. 18, in yet another embodiment of the present invention, the multi-density polymeric interbody spacer 810 includes first density region 812 forming a posterior region 870 of the multi-density polymeric interbody spacer 810 and second density region 814 forming an anterior region 872 of the multi-density polymeric interbody spacer 810. The interface region 836 is formed between the first density region 812 and the second density region 814. The multi-density polymeric interbody spacer 810 may be formed according to the same processes discussed in connection with FIGS. 9, 13 and 17.

Referring to FIG. 19, the multi-density polymeric interbody spacer 910 may also include an axial channel 974 extending axially through the multi-density polymeric interbody spacer 910 from the superior surface 918 to the inferior surface 920. A radial channel 976 extends radially from the spacer perimeter surface 930 into the axial channel 974. The axial channel 974 and the radial channel 976 allow liquid polymer, preferably the biocompatible polymeric material 42, discussed above, to be injected during surgery to achieve a strong adhesive bond between the vertebrae 22, 24 (FIG. 2) and the multi-density polymeric interbody spacer 910.

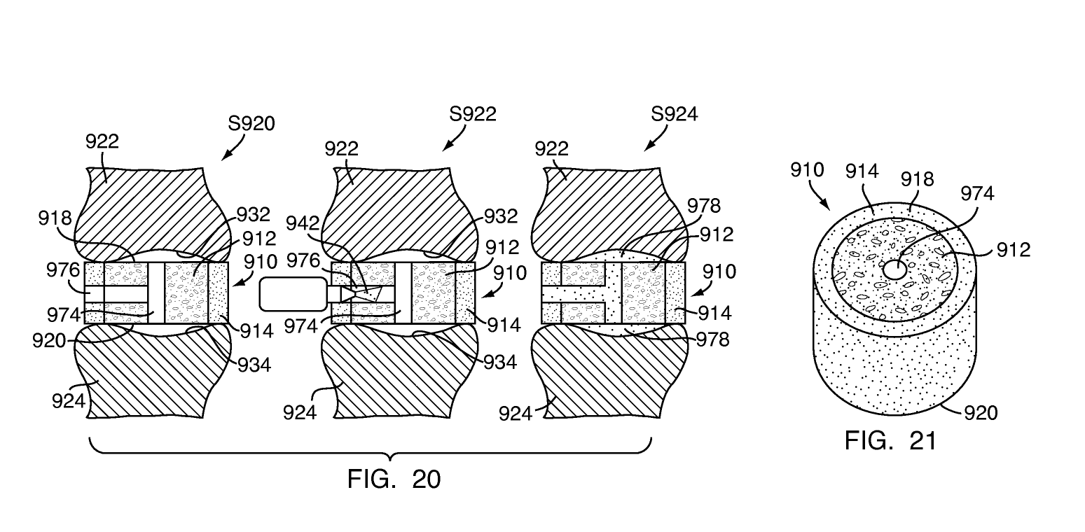

Referring to FIG. 20, in step S920, the multi-density polymeric interbody spacer 910 is placed between the first vertebral end plates 932 and the second vertebral end plate 934. In step S922, the biocompatible polymeric material 942 is injected into radial channel 976. The biocompatible polymeric material 942 passes through the radial channel 976, into the axial channel 974 and extrudes from the multi-density polymeric interbody spacer 910 onto and into the first vertebral end plate 932 and the second vertebral end plate 934. In step S924, the biocompatible polymeric material 942 cures to form an adhesive bond region 978. The adhesive bond region 978 may have the same density as the first density region 912 or the second density region 914. Alternatively, the adhesive bond region 978 may have a density that differs from both the first density region 912 and the second density region 914.

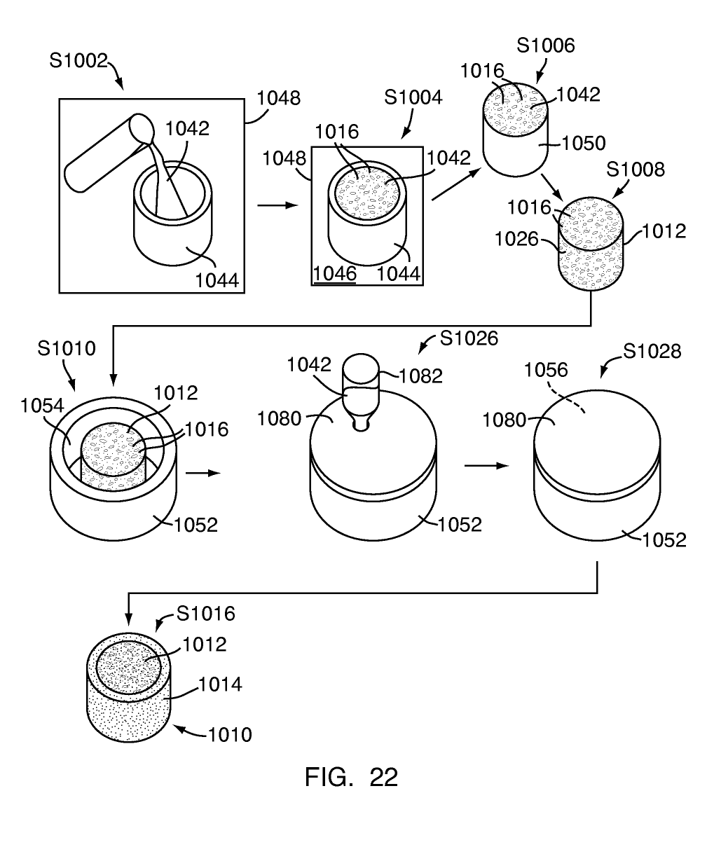

Referring to FIG. 21, the multi-density polymeric interbody spacer 910 may also include the axial channel 974 extending axially through the multi-density polymeric interbody spacer 910 from the superior surface 918 to the inferior surface 920. However, in this embodiment, the multi-density polymeric interbody spacer 910 does not include the radial channel. The axial channel 974 may be filled with an osteoinductive agent including bone morphogenic protein or bone marrow aspirate. The axial channel 974 provides a more direct passageway for cells, nutrients and/or bone to reach the central region of the implanted multi-density polymeric interbody spacer 910.

Referring to FIG. 22, according to another embodiment of the present invention, the lower porosity second region 1014 of the multi-density polymeric interbody spacer 1010 is formed in a closed mold, which provides the second pressure 1056. Steps S1002 through S1008 for forming the first density region 1012 are substantially the same as those discussed in connection with FIG. 9 and, therefore, will not be discussed in further detail. In step S1010, the first density region 1012 is positioned in the second mold 1052, which is a closed mold that may be closed by a mold closure member 1080, i.e. a lid or cover. The second mold 1052 provides space 1054 for producing the second density region 1014. In step S1026, the second mold 1052 is closed with the mold closure member 1080 and biocompatible polymeric material 1042, in liquid state, is injected into the closed second mold 1052 through an injector 1082. The biocompatible polymeric material 1042 may instead be poured into the second mold 1052, as shown in step S12 of FIG. 9, prior to closing the second mold 1052 with the mold closure member 1080. The liquid state biocompatible polymeric material 1042 fills the space 1054 and flows into the pores 1016 formed on the first region perimeter surface 1026 of the first density region 1012. In step S1028, the biocompatible polymeric material 1042 is allowed to polymerize to form the second density region 1014. The closed second mold 1052 restricts expansion of the biocompatible polymeric material 1042 during polymerization, which produces the high-pressure environment and provides the second pressure 1056. Since the expansion of the carbon dioxide byproducts of the polymerization process is restricted, the carbon dioxide produces smaller pores, resulting in a second density region 1014 with a lower porosity and, conversely, a higher density than the first density region 1012. Since the liquid biocompatible polymeric material 1042 is able to flow into the pores 1016 of the first density region 1012 during step S1026, the biocompatible polymeric material 1042 cures in the pores 1016 during step S1028 to form the porous interlocking shown in FIGS. 3 and 4. Preferably, the second pressure 1056 generated within the closed second mold 1052 is in the range of approximately five pounds per square inch to twenty pounds per square inch (5 psi-20 psi) to produce the second density region 1014 having less than approximately fifty percent (50%) porosity. However, the second pressure 1056 may be any pressure capable of forming the desired porosity of the second density region 1014. In step S1016, the fully polymerized biocompatible polymeric material 1042 and the connected first density region 1012 are removed from the second mold 1052 and the polymerized biocompatible polymeric material 1042 is machined to the proper shape of the second density region 1014, if necessary, to form the multi-density polymeric interbody spacer 1010.

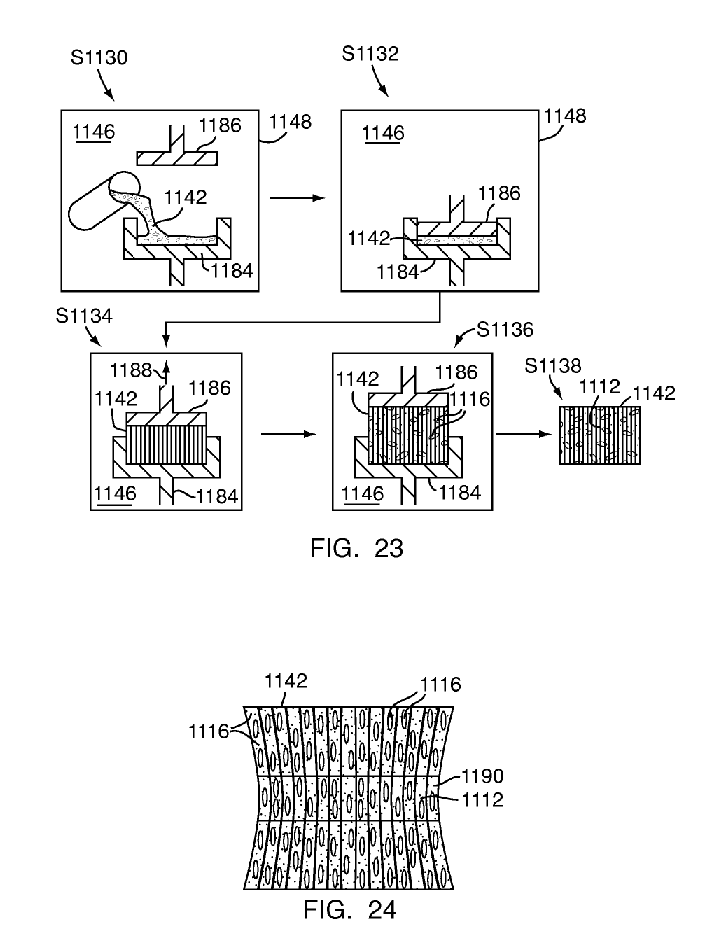

Referring to FIG. 23, in another embodiment of the present invention, the first density region 1112 of the multi-density polymeric interbody spacer may be formed with anisotropic material properties to improve compressive strength and/or tensile strength, without sacrificing the porosity. In step S1130, the biocompatible polymeric material 1142, in liquid state, is poured into a first platen 1184 at the first pressure 1146. When poured, the biocompatible polymeric material 1142 has isotropic material properties. The first platen 1184 is designed to mold the biocompatible polymeric material 1142 into the first density region 1112. The first pressure 1146 may be established, for example, by placing the first platen 1184 in a vacuum chamber 1148 to create a low-pressure environment. In step S1132, a second platen 1186 is lowered onto the liquid biocompatible polymeric material 1142 and held in a fixed position while the biocompatible polymeric material 1142 is allowed to partially cure. For example, the first and second platens 1184, 1186 may be held in the fixed position for approximately five minutes to fifteen minutes (5 minutes-15 minutes) to allow for partial curing. However, the time necessary for partial curing of the biocompatible polymeric material 1142 will largely depend upon the material formulation and may, therefore, vary. Additionally, the curing temperature may also be employed to affect the curing rate and alter the time necessary to fix the first and second platens 1184, 1186. Off-gassing of carbon dioxide byproducts forms large pores 1116 in the same manner discussed in connection with FIG. 9.

In step S1134, when the biocompatible polymeric material 1142 is in the taffy-like stage of the curing process, the first platen 1184 and the second platen 1186 are pulled apart from one another in a displacement direction 1188, thereby pulling the partially cured biocompatible polymeric material 1142, which, therefore, elongates in the displacement direction 1188. For example, the biocompatible polymeric material 1142 may elongate in thickness in the range of approximately fifty percent to three hundred percent (50%-300%) after material expansion due to carbon dioxide release during polymerization. The elongation of the biocompatible polymeric material 1142 results in an anisotropic orientation of the partially cured biocompatible polymeric material 1142. Additionally, the displacement of the first and second platens 1184, 1186 stretches the pores 1116, formed in the taffy-like biocompatible polymeric material 1142, in the displacement direction 1188. In step S1136, the first platen 1184 and the second platen 1186 are held in the displaced position while the biocompatible polymeric material 1142 is maintained at the first pressure 1146 and allowed to fully cure. The taffy-like biocompatible polymeric material 1142 retains its anisotropic orientation while the curing process is completed, which results in anisotropic properties for the fully cured biocompatible polymeric material 1142. As noted above, curing temperatures could affect curing rate. Thus, the anisotropically oriented biocompatible polymeric material 1142 may be formed, in steps S1130 through S1136, at ambient temperature to minimize temperature effects. Alternatively, temperature effects may be exploited by conducting steps S1130 through S1134 at ambient temperature, followed by a rapid heating of the taffy-like biocompatible polymeric material 1142, in step S1136, immediately after the platens are pulled apart, which would quickly cure the biocompatible polymeric material 1142 in the desired structure without risk of the material flowing back into its original shape.

In step S1138, the biocompatible polymeric material 1142 is removed from the first platen 1184 and the second platen 1186. Additionally, the cured biocompatible polymeric material 1142 may be removed from the first pressure 1146. The cured biocompatible polymeric material 1142 may then undergo the remainder of the process of FIG. 9 to remove the skim coat 1150 and/or be shaped to form the first density region 1112.

Referring to FIG. 24, the fully cured anisotropic biocompatible polymeric material 1142 may also have a middle portion 1190 sectioned to form the first density region 1112 if necking, i.e. a localized decrease in cross section of a portion of the biocompatible polymeric material 1142, results from the first and second platens 1184 and 1186 being pulled apart. Even if necking occurs, the middle portion 1190 will retain substantially uniform anisotropic properties.

Additionally, the stretched pores 1116 formed in the cured biocompatible polymeric material 1142 will be oriented longitudinally, providing increased passageways for cell and nutrient migration through the multi-density polymeric interbody spacer (not shown).

Other desirable anisotropic material properties may be achieved by twisting or compressing the first and second platens 1184 and 1186 according to the same process discussed above in connection with FIG. 23. As should be evident to those skilled in the art, the desired properties will depend upon the specific application intended for the multi-density polymeric interbody spacer 1110.

As an alternative to curing the biocompatible polymeric material 1142 in the low-pressure environment discussed in connection with FIG. 23, the desired porosity of the first density region 1112 may also be formed by reticulation, as discussed in connection with FIG. 9.

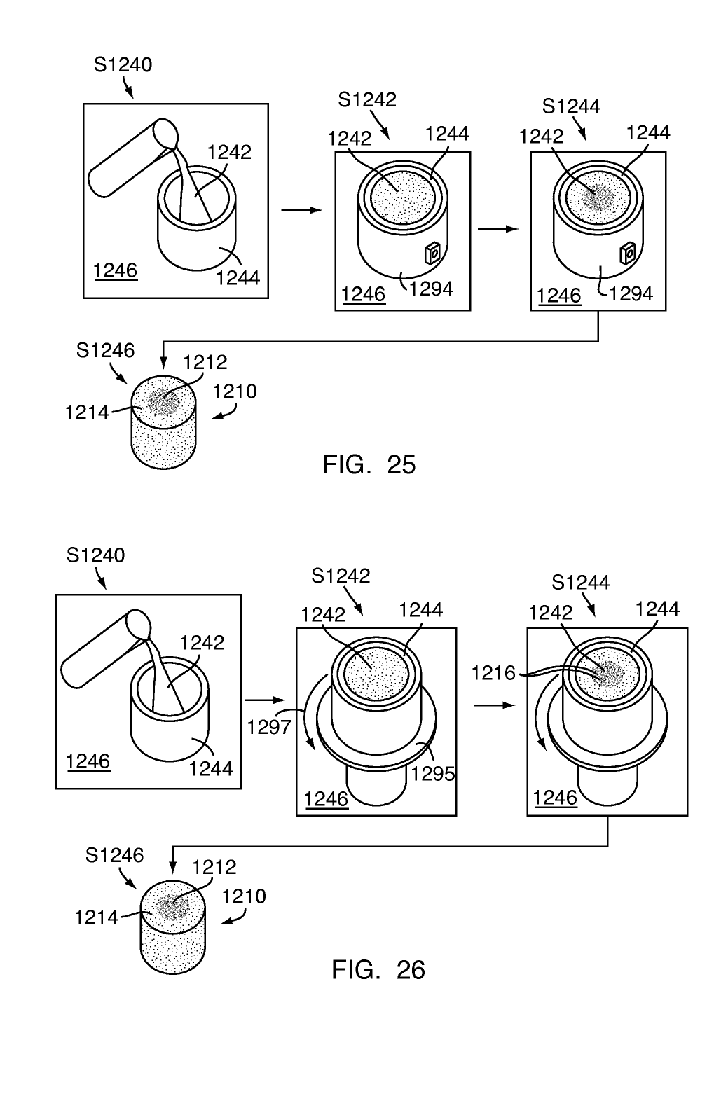

Referring to FIG. 25, a method of simultaneously forming the first and second density regions 1212, 1214 of the multi-density polymeric interbody spacer 1210 is shown. In step S1240, the biocompatible polymeric material 1242, in liquid state, is poured into the first mold 1244 at the first pressure 1246. In step S1242, a temperature control unit 1294, for example a heater, elevates the temperature of the first mold 1244. In step S1244, the biocompatible polymeric material 1242 polymerizes while the elevated temperature and the first pressure 1246 are maintained. A density gradient from the center to the edge of the biocompatible polymeric material 1242 is produced during the polymerization process because the elevated temperature accelerates polymerization near the external surface of the biocompatible polymeric material 1242. The accelerated polymerization results in minimal off-gassing of carbon dioxide, which produces the low porosity second density region 1214. The cooler center of the biocompatible polymeric material 1242 polymerizes more gradually and off-gases carbon dioxide, producing the higher porosity first density region at the core. Preferably, the first pressure 1246 is in the range of approximately ten inches of mercury to thirty inches of mercury (10″ Hg-30″ Hg) to produce the first density region 1212 at the center having approximately sixty percent to ninety percent (60%-90%) porosity. For example, the temperature applied to the first mold 1244 may be greater than one hundred degrees Celsius (100° C.). Additionally, the elevated temperature may be applied to the mold for only a brief time to quickly cure the outer region, but then removed or lowered to allow the core region to cure more slowly. As should be understood by those skilled in the art, both the first pressure 1246 and the curing temperature may be varied to achieve the desired first and second density region 1212,1214 orientation for the multi-density polymeric interbody spacer 1210. In step S1246, the fully polymerized biocompatible polymeric material 1242 is removed from the first mold 1244 to produce the multi-density polymeric interbody spacer 1210. The molding process results in near net production; however, if necessary the multi-density polymeric interbody spacer 1210 may be machined to the proper shape.

Referring to FIG. 26, another embodiment of simultaneously forming the first and second density regions 1212, 1214 of the multi-density polymeric interbody spacer 1210 is shown. In step S1240, the biocompatible polymeric material 1242, in liquid state, is poured into the first mold 1244 at the first pressure 1246. As discussed above, the biocompatible material 1242 may be poured into the first mold 1244 and then subjected to the first pressure 1246. In step S1242, the first mold 1244 is placed in a mold rotation device 1295 and spun at an angular velocity 1297. In step S1244, the biocompatible polymeric material 1242 polymerizes while the angular velocity 1297 is maintained. The angular velocity 1297 produces centrifugal forces that drive carbon dioxide produced during polymerization to the center of the first mold 1244. Thus, a density gradient of pores 1216, from the center to the edge of the biocompatible polymeric material 1242, is produced during the polymerization process. In step S1246, the fully polymerized biocompatible polymeric material 1242 is removed from the first mold 1244 to produce the multi-density polymeric interbody spacer 1210. The molding process results in near net production; however, if necessary the multi-density polymeric interbody spacer 1210 may be machined to the proper shape.

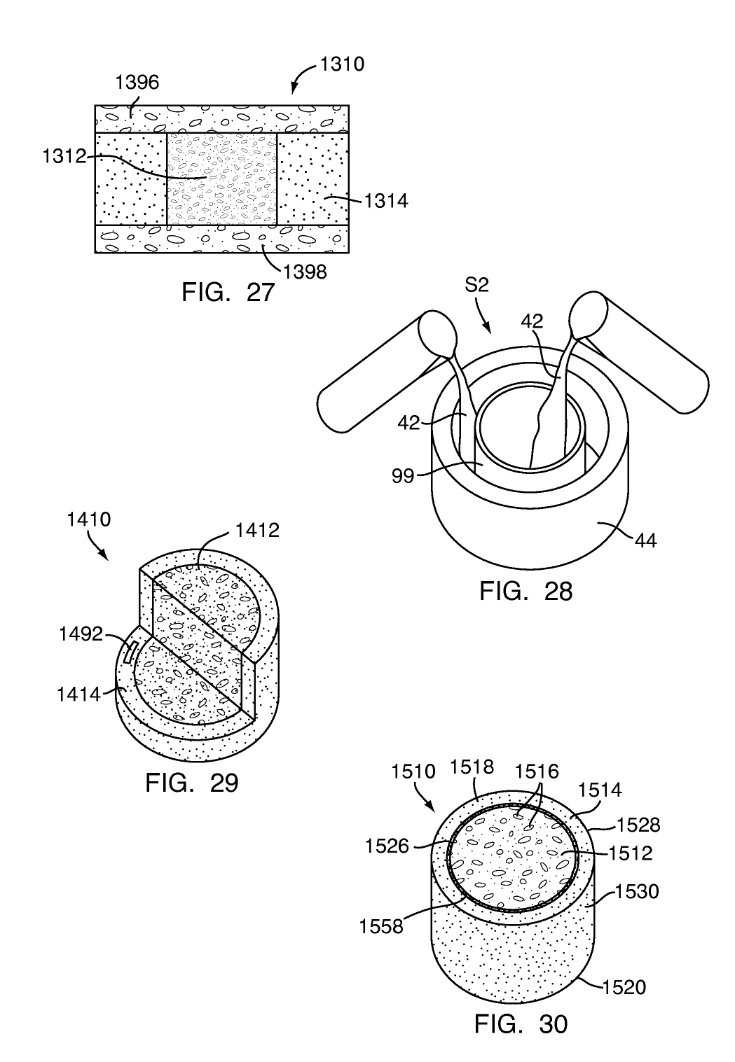

Referring to FIG. 27, another embodiment of the multi-density polymeric interbody spacer 1310, having first and second density regions 1312, 1314, includes a superior porous surface 1396 and an inferior porous surface 1398. As superior surface 1396 and inferior surface 1398 are relatively highly porous, they will partially crush upon implantation between first and second vertebrae (not shown) under the load from the first and second end plates (not shown). This partial crushing forms a custom fit for the multi-density polymeric interbody spacer 1310 between the first and second end plates (not shown).

The first density region 12, 112, 212, 312, 412, 512, 612, 712, 812, 912, 1012, 1112, 1212 and 1312 and second density region 14, 114, 214, 314, 414, 514, 614, 714, 814, 914, 1014, 1114, 1214 and 1314 have been described thus far as having the same formulation with the density of each being dependent upon pressure and/or temperature applied during polymerization or being dependent upon a reticulation procedure. However, the first density region and second density region may instead be formed using biocompatible materials of different formulation. For example, the water concentration of the liquid biocompatible material 42, 942, 1042, 1142 and 1242 used to form the second density region may be decreased from that used to form the first density region. During polymerization, the water in the liquid biocompatible material reacts to produce the carbon dioxide. Therefore, a reduced concentration of water will lead to a smaller production of carbon dioxide and, accordingly, a reduced porosity. Additionally, selecting different biocompatible polymeric materials that are more hydrophilic or more hydrophobic may also alter the formulation and, therefore, the density of the first and second density regions. Similarly, changing the formulation of the biocompatible polymeric material by altering the type or amount of catalyst in the liquid biocompatible material will also change the porosity of the resulting first density region or second density region. The surfactants, polyols and/or prepolymers used to form the liquid biocompatible polymeric material may also be changed to alter the formulation and, in turn, the density of the first density region and the second density region.

One advantage to fabricating multiple density regions, i.e. first density region and second density region, from biocompatible polymeric material with different formulations is that the first and second density regions may be cast simultaneously to achieve the varied densities. Simultaneous casting is possible since the first and second density regions of different formulations do not need to be cured at different first and second pressures, 46, 1046, 1146, 1246, 56 and 1056. The two different formulations of biocompatible polymeric material may be poured into the mold in relatively viscous states, which minimizes the potential for undesirable mixing. Some mixing between the two formulations will still occur at the interface, which will improve connectivity and is, therefore, desirable. Alternatively, referring to FIG. 28, a thin dividing member 99 may be used to initially separate the first and second formulations of biocompatible polymeric material 42 within the mold 44 during the pouring step S2. After pouring, the dividing member 99 may be removed once the formulations have reached a desirable viscosity, allowing the formulations of biocompatible polymeric material 42 to flow against one another and mix at the interface.

The porosity of the first density region and the second density region may also be controlled by mixing technique for preparing the liquid biocompatible polymeric material. For example, mechanical speed mixing, e.g. using a blender, typically results in a uniform pore structure with a small average pore size, while hand mixing typically results in a more random distribution of pore sizes.

Features that have evolved on commercially available interbody spacers may also be implemented in the multi-density polymeric interbody spacer 10 of the present invention. For example, the multi-density interbody spacer 10, 110, 210, 410, 510, 610, 710, 810, 910, 1010, 1210 and 1310 may include bone-contacting surface features such as teeth or cleats or be formed with wedges or angles, as discussed in connection with FIG. 5, to provide proper lordosis. Similarly, the multi-density polymeric interbody spacer may also include known insertion features and/or connection points for instrumentation such as slots, holes, threaded holes, break-off features or undercuts.

Additionally, the multi-density polymeric interbody spacer may include radiolucent markers for assessing position and/or orientation of the multi-density polymeric interbody spacer in vivo. For example, referring to FIG. 29, one or more radiopaque markers 1492 may be cast into the first density region 1412 or second density region 1414 during processing or may be press fit into place. The radiopaque markers 1492 may be needles, rods or a plurality of small beads. The radiopaque markers 1492 may also be injected, in liquid form, into the biocompatible polymeric material while it is in the taffy-like state of the curing process. The injected liquid then cures into solid radiopaque markers 1492 during the remainder of the curing process. Alternatively, barium powder, or other radiopaque powder, may be added to specific regions of the multi-density polymeric interbody spacer during polymerization, allowing the entirety of the specific regions to be viewed in vivo.

The multi-density polymeric interbody spacer of the present invention may also be coated and/or treated with antibiotics and/or an osteoinductive agent to assist in healing and accelerate bone growth after spinal fusion surgery.

Referring to FIG. 30, the various embodiments of the present invention may also implement liquid adhesive to form the third density region 1558 and further improve the direct adhesion and mechanical interlocking disclosed herein. For example, the first and second density regions 1512, 1514 may be formed independently, after which the thin layer of liquid adhesive may be used to form the third density region 1558 bonding the first and second density regions 1512, 1514 together through direct adhesion and porous interlocking. Preferably the liquid adhesive is of the same polyurethane/urea formulation as the first density region 1512 and the second density region 1514. Additionally, during implantation, intraoperative liquid adhesive (not shown) may be applied to the interface between the first end plate 32 and the superior surface 18 and the interface between the second end plate 34 and the inferior surface 20 to enhance adhesion.

Although the present invention has been described as having a denser region formed from polyurethane, the region of greater density may instead be formed of metal. This embodiment differs from prior art spacers with metal outer regions in that the less dense region chemically adheres to the metal portion rather than relying on a press fit between the metal and the less dense region. For example, the KRYPTONITE™ bone matrix product may form the low-density first density region within an outer high-density second density region formed from metal, i.e. steel, titanium, titanium alloy or any similar metal used for surgical implantation, or PEEK.

An advantage of the multi-density polymeric interbody spacer 10, 110, 210, 410, 510, 610, 710, 810, 910, 1010, 1210, 1310, 1410 and 1510 of the present invention is that it provides a structure with the strength to withstand the necessary mechanical loads seen after spinal fusion surgery while also providing a porous structure to promote bone ingrowth.

A further advantage of the present invention is that the method for forming the multi-density polymeric interbody spacer provides for highly reproducible mechanical properties. Whereas cadaver bone varies from sample to sample, spacers of the present invention are fabricated with known and reproducible properties. Additionally, the present invention does not have the storage limitations that accompany cadaver bone spacers. Also, supply of spacers according to the present invention is not limited by available cadaver specimens. Additionally, the size and shape of the multi-density polymeric interbody spacer of the present invention is not restricted by the size and shape of human bone. The multi-density polymeric interbody spacer also eliminates the risk of disease transfer associated with many prior art interbody spacers.

Another advantage of the present invention is that the multi-density polymeric interbody spacer may be formed to customized shapes and geometries for different bone fusion applications. Additionally, the multi-density polymeric interbody spacer of the present invention may incorporate a variety of surface features to improve fit between and contact with first and second vertebrae.

A further advantage of the present invention is that the multi-density polymeric interbody spacer is compatible with know insertion features meaning that no additional tooling is required for implantation.

Although this invention has been shown and described with respect to the detailed embodiments thereof, it will be understood by those skilled in the art that various changes in form and detail thereof may be made without departing from the spirit and the scope of the invention. For example, although the multi-density polymeric interbody spacer has been described as a spacer for spinal fusion surgery, the multi-density polymeric interbody spacer may also be configured for other orthopedic applications such as fusion of critical defects in long bones.

What is claimed is:

1. A multi-density polymeric interbody spacer comprising:
a first density region of a first biocompatible polymeric material of a first porosity;
a second density region of a second biocompatible polymeric material of a second porosity; and
an interface region between the first density region and the second density region;
wherein the first density region and the second density region are connected to one another in the interface region by direct adhesion and porous interlocking.

2. The multi-density polymeric interbody spacer according to claim 1, wherein the first density region forms an inner core and the second density region forms an outer region substantially surrounding the first density region.

3. The multi-density polymeric interbody spacer according to claim 2, wherein the second density region has a greater density than the first density region.

4. The multi-density polymeric interbody spacer according to claim 1, additionally comprising a third density region of a third porosity connected to at least one of the first or second density regions by direct adhesion and porous interlocking.

5. The multi-density polymeric interbody spacer according to claim 4, wherein the third porosity is substantially the same as the first porosity or the second porosity.

6. The multi-density polymeric interbody spacer according to claim 4, wherein the third porosity is different than the first porosity and the second porosity.

7. The multi-density polymeric interbody spacer according to claim 4, wherein the second density region and the third density region are laterally disposed on either side of the medial first density region.

8. The multi-density polymeric interbody spacer according to claim 7, wherein two of the density regions have substantially the same porosity.

9. The multi-density polymeric interbody spacer according to claim 1, wherein the second porosity is less than the first porosity.

10. The multi-density polymeric interbody spacer according to claim 1, wherein the first biocompatible polymeric material and the second biocompatible polymeric material are of substantially equivalent chemical formulation.

11. The multi-density polymeric interbody spacer according to claim 1, wherein the first porosity is in the range of approximately sixty percent to ninety percent.

12. The multi-density polymeric interbody spacer according to claim 1, wherein the second porosity is less than approximately fifty percent.

13. The multi-density polymeric interbody spacer according to claim 1, wherein the first density region has anisotropic properties.

14. The multi-density polymeric interbody spacer according to claim 1, wherein the first density region and the second density region form superior and inferior surfaces for contacting first and second vertebral end plates and wherein at least one of said surfaces includes a surface feature to minimize spacer migration.

15. The multi-density polymeric interbody spacer according to claim 1, wherein the first density region forms a posterior region of the multi-density polymeric interbody spacer and the second density region forms an anterior region of the multi-density polymeric interbody spacer.

16. The multi-density polymeric interbody spacer according to claim 1, wherein a spacer perimeter has a substantially trapezoidal shape.

17. The multi-density polymeric interbody spacer according to claim 1, additionally comprising a porous superior surface and a porous inferior surface for partial crushing to form a custom fit between first and second vertebral end plates.

18. The multi-density polymeric interbody spacer according to claim 1, including an axial channel extending through the multi-density polymeric interbody spacer.

19. The multi-density polymeric interbody spacer according to claim 18, including a radial channel extending from a spacer perimeter surface to the axial channel.

20. The multi-density polymeric interbody spacer according to claim 1, additionally comprising a radiopaque marker for assessing orientation of the multi-density polymeric interbody spacer.

21. The multi-density polymeric interbody spacer according to claim 20, wherein the radiopaque marker is cast within the multi-density polymeric interbody spacer.

22. The multi-density polymeric interbody spacer according to claim 21, wherein the radiopaque marker includes radiopaque material as a filler dispersed within at least one of the first and second density regions.

23. The multi-density polymeric interbody spacer according to claim 1, wherein at least one of the first or second biocompatible polymeric materials includes an antibiotic to assist in healing after surgery.

24. The multi-density polymeric interbody spacer according to claim 1, wherein at least one of the first or second biocompatible polymeric materials includes an osteoinductive agent to accelerate bone growth after surgery.

25. The multi-density polymeric interbody spacer according to claim 1, wherein the first biocompatible polymeric material and the second biocompatible polymeric material are of different chemical formulation.

26. The multi-density polymeric interbody spacer according to claim 25, wherein at least one of the biocompatible polymeric materials is substantially hydrophilic.

27. A multi-density interbody spacer comprising:
a first density region of a first biocompatible polymeric material; and
a second density region of a second biocompatible polymeric material;
wherein an interface region is formed between the first density region and the second density region having mechanical interlocking.

28. The multi-density interbody spacer according to claim 27, wherein the mechanical interlocking includes porous interlocking formed by said second biocompatible polymeric material partially invading the first density region.

29. The multi-density interbody spacer according to claim 27, wherein the mechanical interlocking includes a macro feature.

30. A multi-density polymeric interbody spacer comprising:
a first density region with a first porosity;
a second density region of a second porosity formed adjacent to the first density region during surgery.

31. A multi-density interbody spacer comprising:
a first density region of a first biocompatible polymeric material;
a second density region of a second biocompatible polymeric material; and
an interface region between the first and second density regions.

32. The multi-density spacer according to claim 31, wherein the interface region includes mixing between the first biocompatible polymeric material and the second biocompatible polymeric material.

33. The multi-density spacer according to claim 32, wherein the interface region includes direct adhesion and mechanical interlocking.

34. The multi-density spacer according to claim 31, wherein the interface region includes direct adhesion.

35. The multi-density interbody spacer according to claim 31, wherein the interface region includes a third density region formed from a thin layer of liquid adhesive bonding with the first and second density regions.

36. The multi-density interbody spacer according to claim 31, additionally comprising at least one insertion feature easing implantation and handling of the multi-density interbody spacer.

37. The multi-density interbody spacer according to claim 36, wherein the at least one insertion feature includes lateral slots.
